(19) United States
(12) Patent Application Publication
(10) Pub. No.: US 2014/0169527 A1
(43) Pub. Date: Jun. 19, 2014

(54) PHOTOACOUSTIC DETECTOR, PHOTOACOUSTIC BOARD AND DETECTOR USING THE PHOTOACOUSTIC BOARD

(71) Applicant: INDUSTRIAL TECHNOLOGY RESEARCH INSTITUTE, (US)

(72) Inventors: Shih-Bin Luo, Hsinchu County (TW); De-Yi Chiou, New Taipei City (TW); Chi-Shen Chang, Hsinchu County (TW); Wan-Ting Tien, Chiayi City (TW); Kuo-Tung Tiao, Hsinchu County (TW)

(73) Assignee: INDUSTRIAL TECHNOLOGY RESEARCH INSTITUTE, Hsinchu (TW)

(21) Appl. No.: 13/831,922

(22) Filed: Mar. 15, 2013

(30) Foreign Application Priority Data
Dec. 17, 2012 (TW) 101224436

(51) Int. Cl. G01N 21/17; A61B 6/00

(52) U.S. Cl. CPC G01N 21/1702; A61B 6/4266
USPC 378/63; 73/655

(57) ABSTRACT

A photoacoustic detector, a photoacoustic board and a detector using the photoacoustic board are provided. The photoacoustic detector includes a X-ray transmitter, a X-ray receiver, a light module and an ultrasonic module. The X-ray transmitter is configured to transmit a X-ray to irradiate the object. The X-ray receiver is configured to receive an image beam generated after the object is irradiated by the X-ray. The light module is configured to provide light for illuminating the object to generate a first sonic wave signal. The sonic module is configured to receive the first sonic wave signal, transmit an sonic wave toward the object, and receive a second sonic wave signal generated after the object is interacted with the sonic wave. Therefore, a X-ray image, an ultrasonic image and a photoacoustic image are obtained via the photoacoustic detector.

### CROSS-REFERENCE TO RELATED APPLICATION

This application claims the priority benefit of Taiwan application serial no. 101224436, filed on Dec. 17, 2012. The entirety of the above-mentioned patent application is hereby incorporated by reference herein and made a part of this specification.

### TECHNICAL FIELD

The technical field generally relates to a detector, and more particularly, to a photoacoustic detector, a photoacoustic board and a detector using the photoacoustic board.

### BACKGROUND

Breast cancer is one of the cancers which likely occur on women. A calcified tissue in breasts is an important feature to detect breast cancers in the early stage. In the current detecting procedure of breast cancer, the first step is taking X-ray photos on breasts. The image contrast of X-ray photos is good, but it may cause some concerns about getting caners due to the radiation, therefore the detection cannot be done frequently. On the other hand, if the breasts to be detected are dense, then the pressure on the breasts may cause severe pain because the breasts need to be fixed when taking X-ray photos. In addition, soft tissues cannot be detected by X-ray photographing and a X-ray image cannot be rendered in real time. After taking a X-ray photo on breasts and being diagnosed by a doctor, it may further needs ultrasonic photographing for the woman being detected. Soft tissues can be detected by ultrasonic photographing and the images could be rendered in real time. But, the images captured by ultrasound photographing have speckle noises, which may make the contrast of the images bad. And, the technical demand for an operator to do the ultrasound photographing is relatively more.

Therefore, it is an important issue concerned by related technicians to ease the uncomfortable feeling of the person being detected, and to provide different diagnosing results at the same time.

Nothing herein should be construed as an admission of knowledge in the prior art of any portion of the present disclosure. Furthermore, citation or identification of any document in this application is not an admission that such document is available as prior art to the present disclosure, or that any reference forms a part of the common general knowledge in the art.

### SUMMARY

An exemplary embodiment provides a photoacoustic detector comprising a X-ray transmitter, a X-ray receiver, a light module and a sonic module. The X-ray transmitter transmits a X-ray to irradiate the object. The X-ray receiver receives an image beam generated after the object is irradiated by the X-ray. The light module provides light for illuminating the object to generate a first sonic wave signal. The sonic module receives the first sonic wave signal, emits a sonic wave toward the object, and receives a second sonic wave signal generated after the object is interacted with the sonic wave.

According to another exemplary embodiment, a photoacoustic board is provided. The photoacoustic board includes optical units and ultrasonic transducers. The optical units and the ultrasonic transducers are disposed on the photoacoustic board in an array arrangement.

According to another exemplary embodiment, a detector using the said photoacoustic board is provided.

It should be understood, however, that this Summary may not contain all of the aspects and embodiments of the present disclosure, is not meant to be limiting or restrictive in any manner, and that the disclosure as disclosed herein is and will be understood by those of ordinary skill in the art to encompass obvious improvements and modifications thereto.

These and other exemplary embodiments, features, aspects, and advantages of the disclosure will be described and become more apparent from the detailed description of exemplary embodiments when read in conjunction with accompanying drawings.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram illustrating a photoacoustic detector according to an exemplary embodiment.

FIGS. 2A, 3A and 4A illustrate top views of photoacoustic boards according to an exemplary embodiment.

FIGS. 2B, 3B, and 4B illustrate front views of photoacoustic boards according to an exemplary embodiment.

FIGS. 5A and 5B are schematic diagrams illustrating the disposition of photoacoustic boards according to an exemplary embodiment.

FIG. 6A is a schematic diagram illustrating a lens and an actuator in an optical unit according to an exemplary embodiment.

FIGS. 6B and 6C are schematic diagrams illustrating that a lens and an actuator are disposed in every optical unit on a photoacoustic board according to an exemplary embodiment.

FIG. 6D˜6F are schematic diagrams illustrating the adjustment of the angle of light illuminating the object according to an exemplary embodiment.

FIG. 7 is a schematic diagram illustrating a transparent ultrasonic sensor according to an exemplary embodiment.

FIG. 8 is a schematic diagram illustrating a flexible light guide film according to an exemplary embodiment.

FIG. 9 is a schematic diagram illustrating the using of a flexible light guide film and a board-shape ultrasonic transducer according to an exemplary embodiment.

FIGS. 10A and 10B are schematic diagrams illustrating the using of a ultrasonic probe according to an exemplary embodiment.

FIG. 11 is a schematic diagram illustrating a X-ray image, an ultrasonic image and a photoacoustic image according to an exemplary embodiment.

### DESCRIPTION OF THE EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

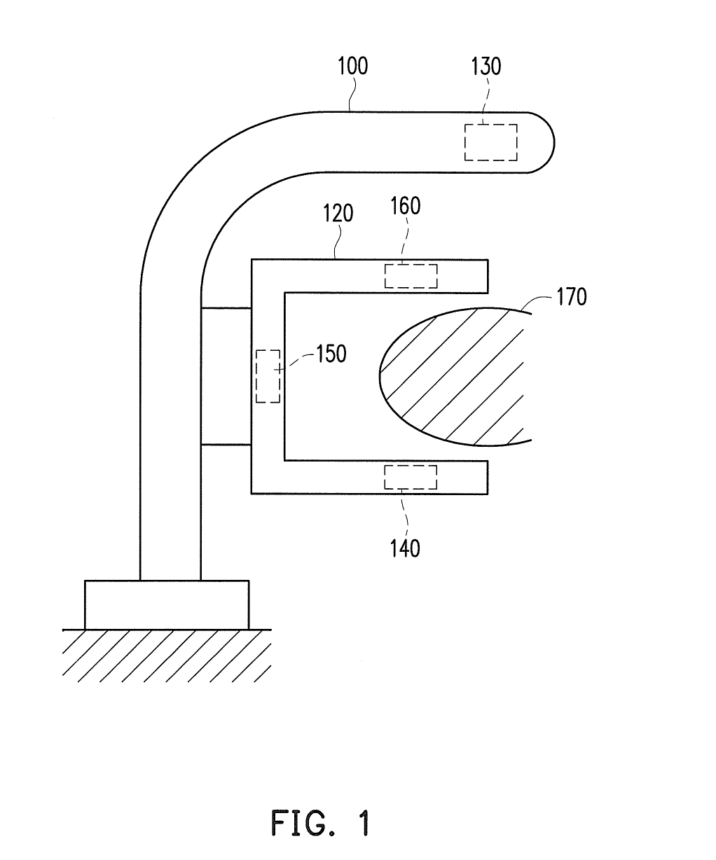

FIG. 1 is a schematic diagram illustrating a photoacoustic detector according to an exemplary embodiment.

Referring to FIG. 1, a photoacoustic detector 100 includes a fixing unit 120, a X-ray transmitter 130, a X-ray receiver 140, a light module 150 and a sonic module 160. The photoacoustic detector 100 is used for detecting an object 170 and obtaining a X-ray image, an ultrasonic image and a photoacoustic image of the object 170. In one exemplary embodiment, the object 170 is a breast. However, the object 170 may be other biological or non-biological tissues, which is not limited in the disclosure.

To be specific, the fixing unit 120 is used for fixing the object 170. In one exemplary embodiment, the fixing unit 120 includes a first pressing board and a second pressing board, which are connected to a movable structure. The positions of the first pressing board and the second pressing board are changed via the movable structure and the first pressing board and the second pressing board clamp the object 170. However, the fixing unit 120 may be at least one hemisphere-shape mask in other exemplary embodiments. Alternatively, the fixing unit 120 may include a cavity for putting the object 170 on. The disclosure does not limit the shape and the structure of the fixing unit 120. In another exemplary embodiment, the fixing unit 120 may be omitted, and the X-ray transmitter 130, the X-ray receiver 140, the light module 150 and the sonic module 160 may be disposed on another component of the photoacoustic detector 100, which is not limited in the disclosure.

The X-ray transmitter 130 transmits a X-ray to irradiate the object 170. And the X-ray receiver 140 receives an image beam generated after the object 170 is irradiated by the X-ray. The image beam is used to generate a two-dimensional X-ray image. Medical personnel may use the X-ray image to detect if there is a calcified point.

The light module 150 provides light to illuminate the object 170 to result in a thermoelastic effect inside the object 170, and therefore a first sonic wave signal is generated. The sonic module 160 receives the first sonic wave signal for generating a photoacoustic image. Herein, the frequency range of the first sonic wave signal includes ranges of ultrasounds or regular sounds, which is not limited in the disclosure.

In addition, the sonic module 160 may transmit a sonic wave toward the object 170 and receive a second sonic wave signal generated after the object 170 is interacted with the sonic wave. The second sonic wave signal is used for generating a sonic image. Herein, the frequency range of the second sonic wave signal may include ranges of ultrasounds or regular sounds, which is not limited in the disclosure.

Note that the light module 150 may include a lighting unit, a light guide, a flexible light guide film, an optical board, a lens or an actuator; these units are configured to provide the light illuminating the object 170. The sonic module 160 may include a sonic transducer, a sonic probe, a transparent ultrasonic sensor, or a board-shape ultrasonic transducer; these units are configured to receive the first sonic wave signal, provide the second sonic wave signal or receive the second sonic wave signal. For example, the ultrasonic transducer may be a capacitive micromachined ultrasonic transducer (CMUT), a polyvinylidene fluoride (PVDF) transducer, a CdZnTe (CZT) transducer, a piezoelectric transducer (PZT), a piezoelectric micro-machined ultrasonic transducer (pMUT) or a combination thereof. The lighting unit may be a solid-state light source or a diode laser. Alternatively, the light module 150 and the sonic module 160 may be implemented together as a photoacoustic board. The disclosure does not limit the units included in the light module 150 and the sonic module 160. In addition, the disclosure does not limit the disposed positions of the fixing unit 120, the X-ray transmitter 130, the X-ray receiver 140, the light module 150 and the sonic module 160. Several exemplary embodiments will be provided to describe different implementations of the photoacoustic detector 100.

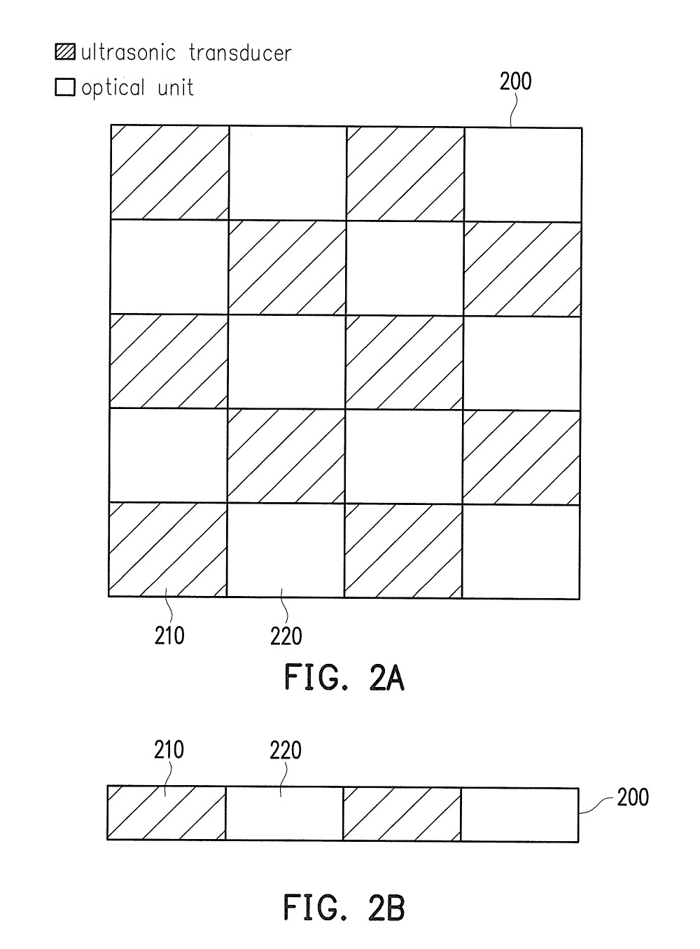

FIGS. 2A, 3A and 4A illustrate top views of photoacoustic boards according to an exemplary embodiment. FIGS. 2B, 3B, and 4B illustrate front views of photoacoustic boards according to an exemplary embodiment.

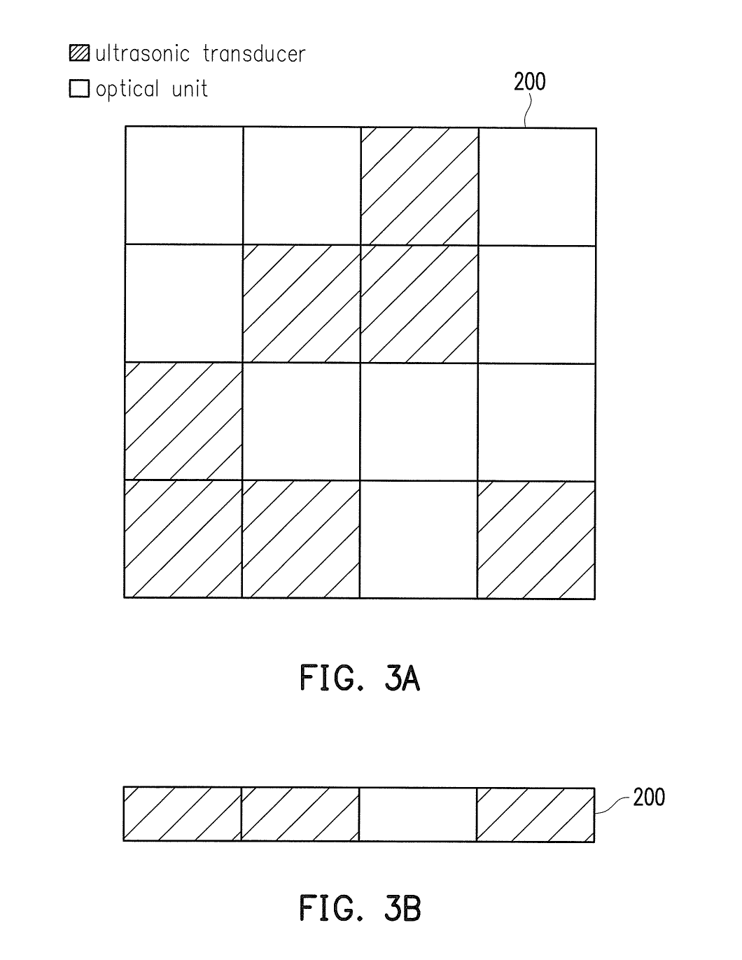

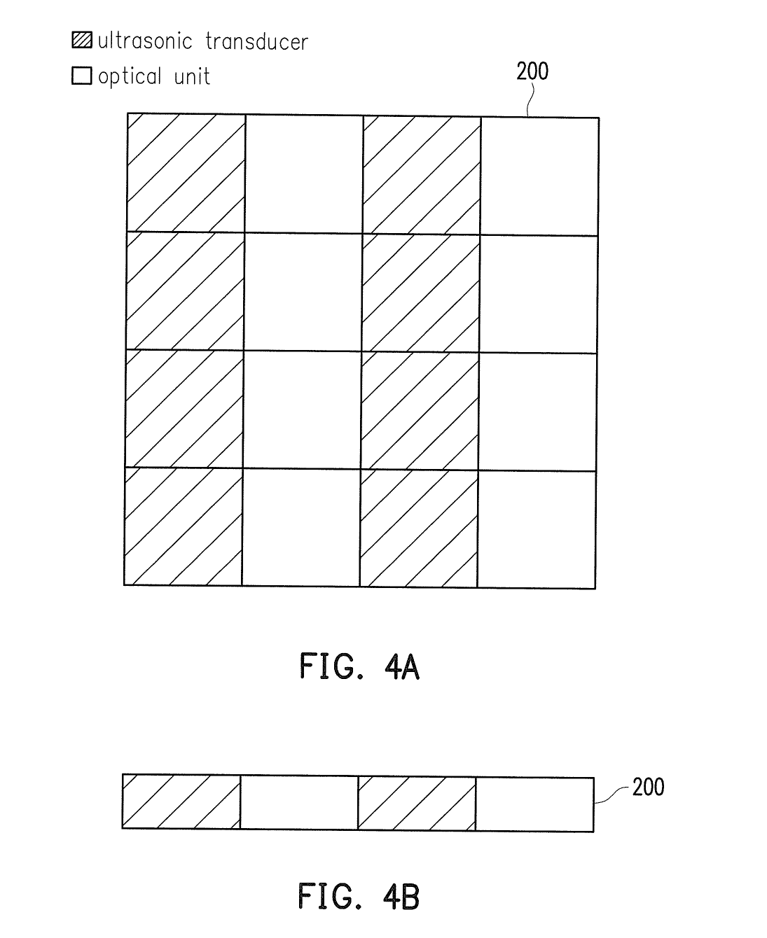

Referring to FIGS. 2A and 2B, in one exemplary embodiment, the light module 150 includes a plurality of optical units, and the optical units may be lighting units, light guides or the combination thereof. If an optical unit is the lighting unit, the optical unit itself is capable of providing light to illuminate the object 170. If an optical unit is the light guide, the optical unit guides light from other lighting units to the object 170. On the other hand, the sonic module 160 includes a plurality of ultrasonic transducers. The optical units (e.g. optical unit 220) and the ultrasonic transducers (e.g. ultrasonic transducer 210) are disposed on a photoacoustic board 200 in an array arrangement. The optical units and the ultrasonic transducers are disposed on the photoacoustic board 200 in a chessboard arrangement in the exemplary embodiment illustrated in FIGS. 2A and 2B. However, the optical units and the ultrasonic transducers are disposed on the photoacoustic board 200 in a random arrangement (illustrated in FIGS. 3A and 3B) in another exemplary embodiment. Alternatively, the optical units and the ultrasonic transducers are disposed on the photoacoustic board 200 in a line arrangement (illustrated in FIGS. 4A and 4B). The disclosure does not limit the arrangements of the optical units and the ultrasonic transducers. And, the number of the optical units may be different from the number of the ultrasonic transducers on the photoacoustic board, which is not limited in the disclosure.

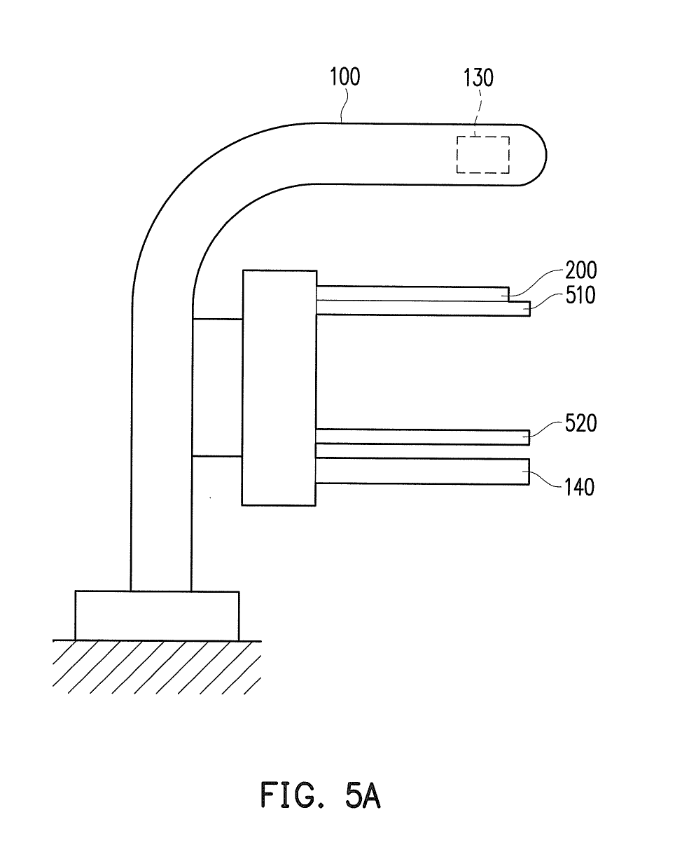

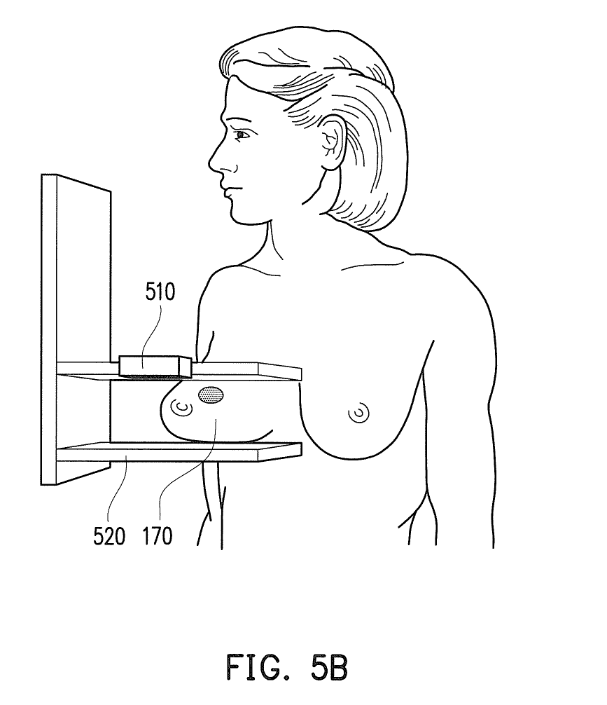

FIGS. 5A and 5B are schematic diagrams illustrating the disposition of photoacoustic boards according to an exemplary embodiment.

Referring to FIGS. 5A and 5B, in one exemplary embodiment, the fixing unit 120 includes a pressing board 510 and a pressing board 520. The photoacoustic board 200 is disposed at a side of the pressing board 510. However, in other embodiments, the photoacoustic board 200 may be disposed at a side of the pressing board 520. Alternatively, the light module 150 and the sonic module 160 may be implemented together as at least one photoacoustic board. If the light module 150 and the sonic module 160 are implemented together as two photoacoustic boards, then one of the photoacoustic board is disposed at a side of the pressing board 510, and the other one is disposed at the other side of the pressing board 520 (between the pressing board 520 and the X-ray receiver 140). The disclosure does not limit the number and the disposed positions of the photoacoustic boards. Herein, one of the pressing board 510 and the pressing board 520 is referred as a first pressing board, and the other one is referred as a second pressing board.

In the exemplary embodiment illustrated in FIG. 5A, the photoacoustic board 200 is removable. When obtaining the X-ray image, the photoacoustic board 200 is not disposed on the pressing board 510. After (or before) obtaining the X-ray image, medical personnel may put the photoacoustic board 200 on the pressing board 510, and the photoacoustic board 200 transmits or guides light toward the object 170, such that the thermoelastic effect is resulted in the tissue of the object 170. The photoacoustic board 200 receives the sonic wave signal generated due to the thermoelastic effect to produce a photoacoustic image. Besides, the photoacoustic board 200 may transmit a sonic wave toward the object 170 and receive the sonic wave signal generated after the object 170 is interacted with the sonic wave to obtain an ultrasonic image. However, the photoacoustic board 200, and the pressing board 510 or pressing board 520 are integrated in another exemplary embodiment. In other words, the optical units and the ultrasonic transducers are disposed on the pressing board 510 or the pressing board 520. As a result, the X-ray image, the ultrasonic image and the photoacoustic image are obtained at the same time.

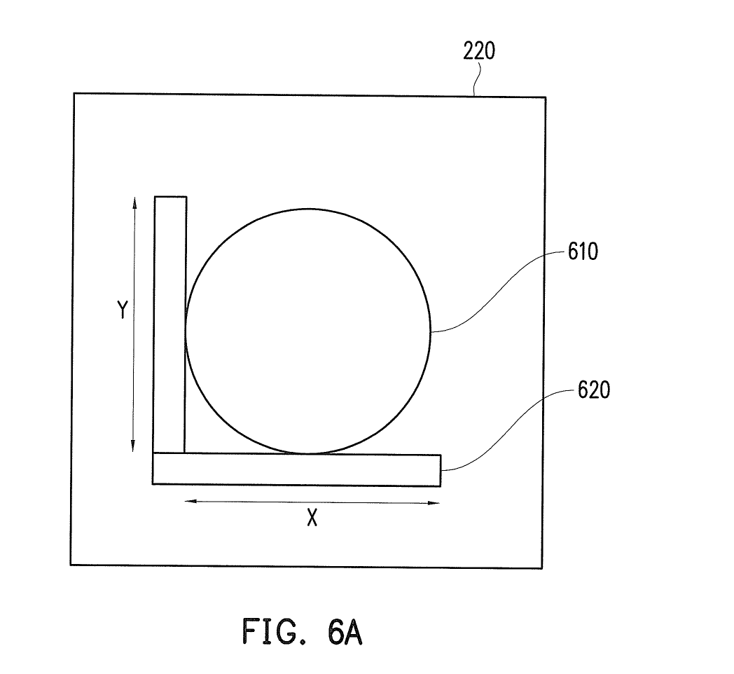

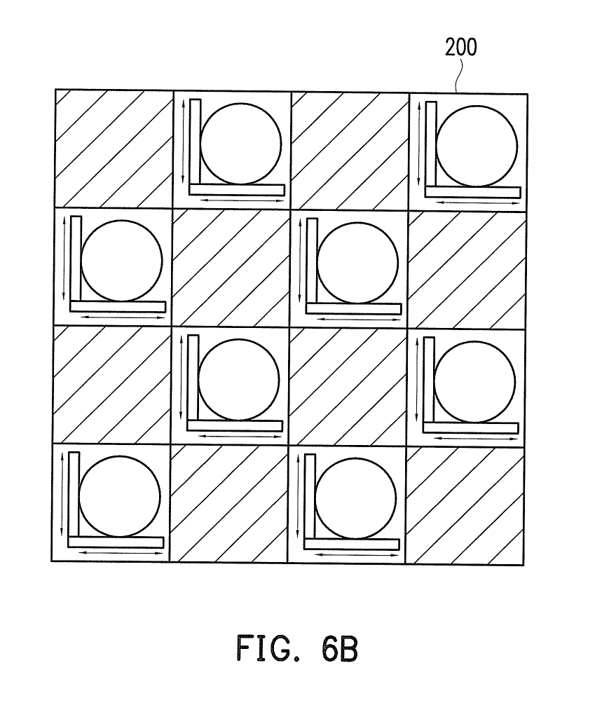

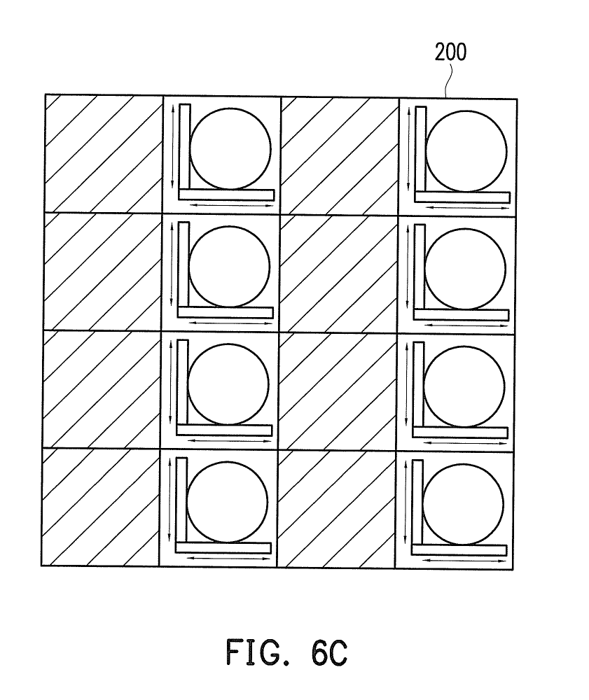

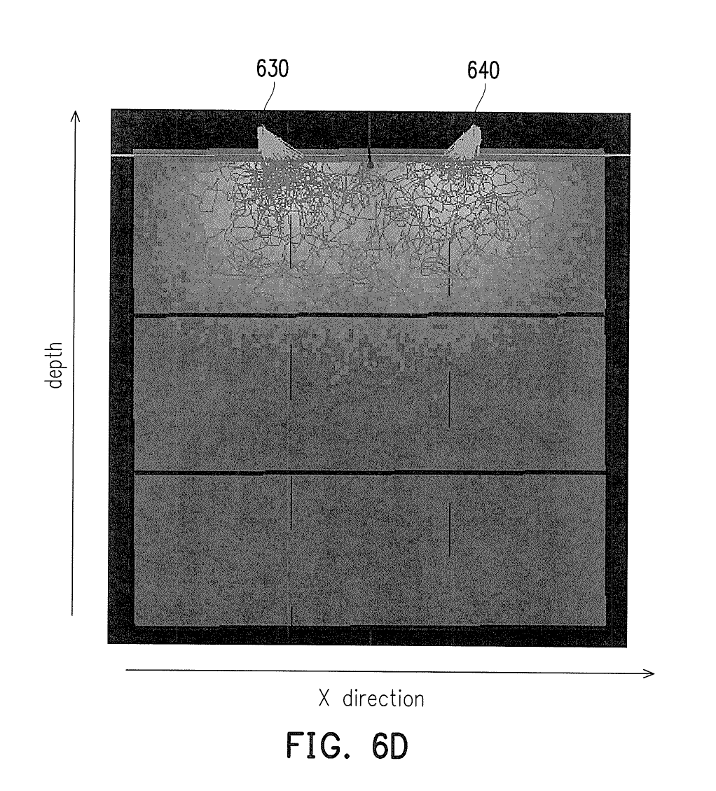

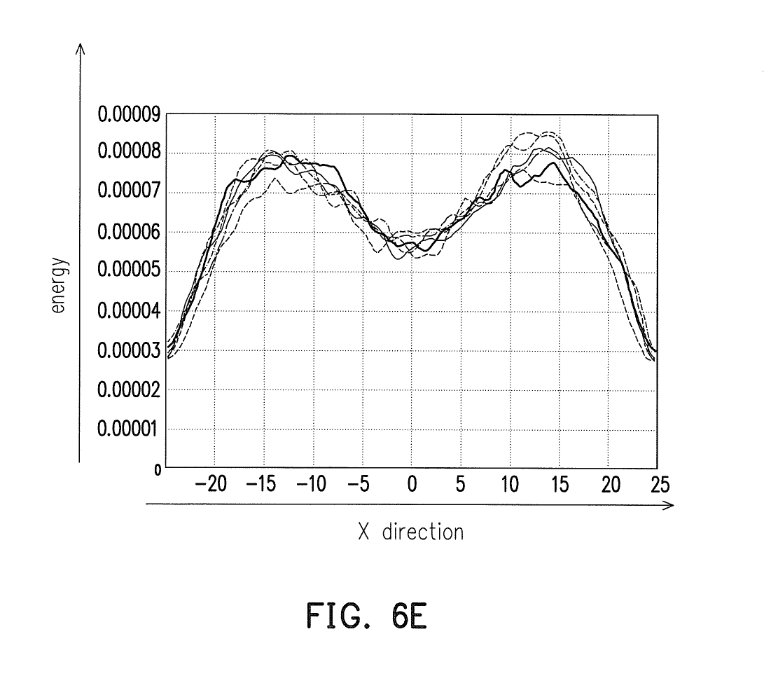

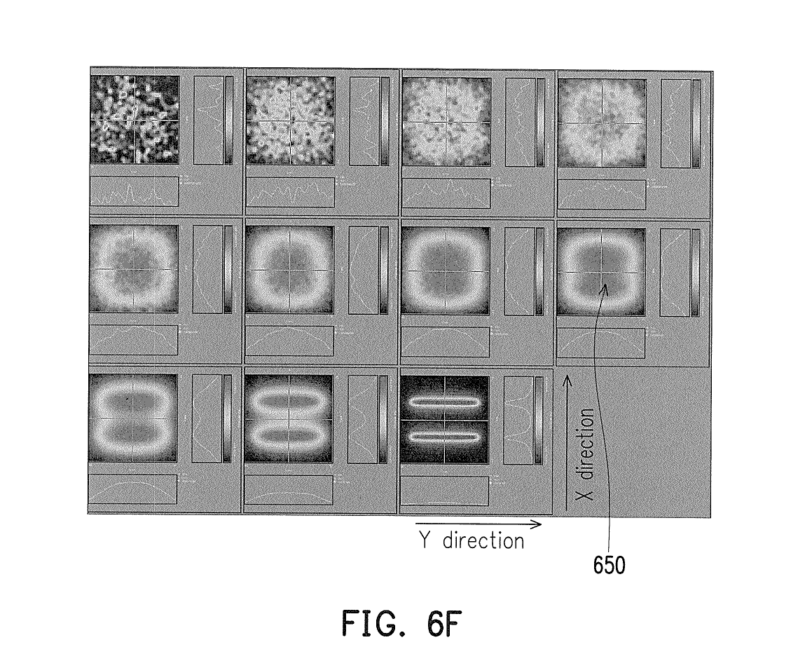

FIG. 6A is a schematic diagram illustrating a lens and an actuator in an optical unit according to an exemplary embodiment.

Referring to FIG. 6A, in one exemplary embodiment, an optical unit on the photoacoustic board further includes a lens and an actuator. For example, the optical unit 220 includes the lens 610 and the actuator 620. The light provided by the optical unit 220 passes through the lens 610 to illuminate the object. The actuator 620 is used for changing the position of the lens 610 in X direction or Y direction. Therefore the angle of the light transmitted from the optical unit 220 is changed, and the angle that the light illuminates the object is changed. In one exemplary embodiment, the position of the lens 610 on the photoacoustic board 200 is adjusted so that the light transmitted from the photoacoustic board 200 uniformly illuminates the object.

FIGS. 6B and 6C are schematic diagrams illustrating that a lens and an actuator are disposed in every optical unit on a photoacoustic board according to an exemplary embodiment. However, the disclosure does not limit that the number of the lens is necessary to be consistent with the number of the actuator on the photoacoustic board.

FIG. 6D˜6F are schematic diagrams illustrating the adjustment of the angle of light illuminating the object according to an exemplary embodiment.

In one exemplary embodiment, every optical unit on the photoacoustic board includes a lens and an actuator (e.g. implemented as the photoacoustic board illustrated in FIGS. 6B and 6C). And, the angle of the light illuminating the object provided by every optical unit may be different with each other. Therefore, in the exemplary embodiments illustrated in FIGS. 6B and 6C, the energy of the light illuminating the object is controlled to be uniform. For example, referring to FIGS. 6D and 6E, there is uniform energy in X direction because the angle of light 630 and light 640 transmitting toward the object is changeable. Please refer to FIG. 6F, wherein some energy schematic maps of different depths are illustrated. For example, in the energy schematic map 650 of a certain depth, light uniformly illuminates the object no matter in X direction or Y direction.

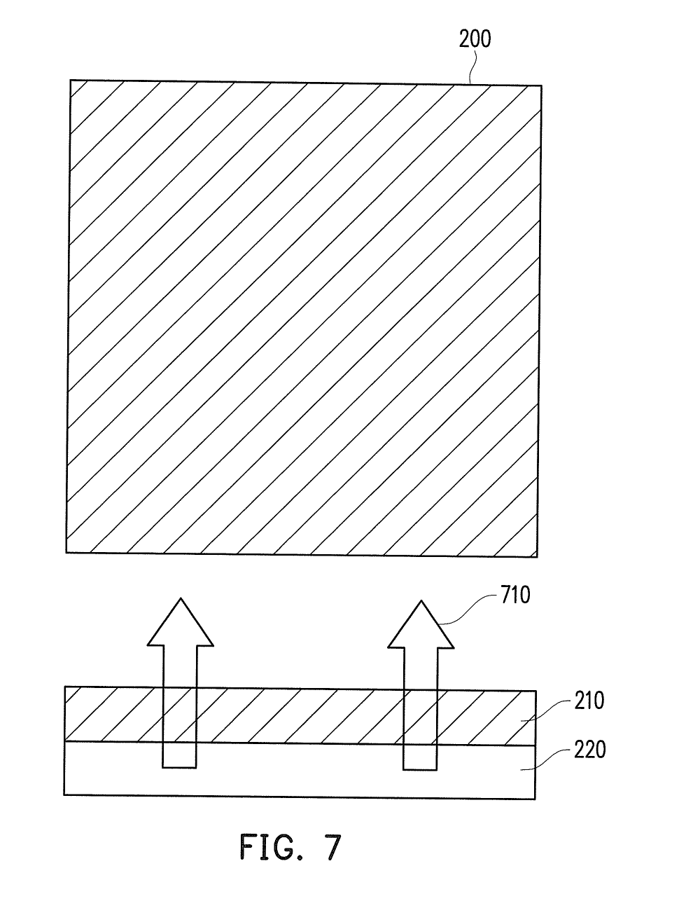

FIG. 7 is a schematic diagram illustrating a transparent ultrasonic sensor according to an exemplary embodiment.

Referring to FIG. 7, in one exemplary embodiment, an ultrasonic transducer on the photoacoustic board 200 is a transparent ultrasonic sensor. The transparent ultrasonic sensor is disposed on an optical unit and the transparent ultrasonic sensor is transparent for the light provided by the optical unit. For example, the light provided by the optical unit 220 passes through the transparent ultrasonic sensor (i.e. the ultrasonic transducer 210) and illuminates the object 170. For example, the transparent ultrasonic sensor includes a transparent film. The material of the transparent film includes a polymer material, silicon (Si), quartz (SiO₂), silicon nitride (Si₃N₄), Al₂O₃, a single crystal material, and other materials that allow light having the wavelength of 10˜2400 nanometers to pass through. The aforementioned polymer material comprises at least one of benzocyclobutene (BCB), polyimide (PI), epoxy photoresist SU8, polydimethylsiloxane (PDMS), and other suitable polymer materials.

In one exemplary embodiment, any implementation of the aforementioned photoacoustic board can be solely used in a detector. And, the disclosure does not limit whether the detector includes a X-ray transmitter and a X-ray receiver or not. When using the detector, medical personnel may obtain a photoacoustic image and an ultrasonic image via the photoacoustic board in the detector. However, each implementation of the photoacoustic board has been described in detail above, which is not repeated.

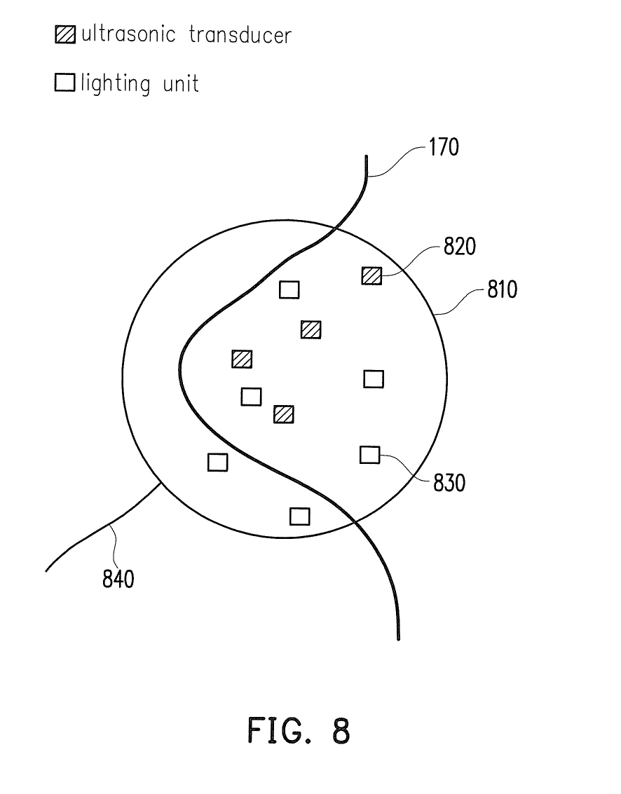

FIG. 8 is a schematic diagram illustrating a flexible light guide film according to an exemplary embodiment.

Referring to FIG. 8, in one exemplary embodiment, the light module 150 includes a lighting unit and a flexible light guide film 810. The flexible light guide film 810 covers the object 170 and guide the light transmitted from the lighting unit. In one exemplary embodiment, there is a distance between the lighting unit and the flexible light guide film 810, and the light provided by the lighting unit illuminates the flexible light guide film 810. However, in another exemplary embodiment, the light transmitted from the lighting unit passes through the light guide 840 to the flexible light guide film 810, which is not limited in the disclosure.

In one exemplary embodiment, the sonic module 160 includes a plurality of ultrasonic transducers. The ultrasonic transducers (e.g. the ultrasonic transducer 820) are disposed on the flexible light guide film 810. And, several lighting units (e.g. the lighting unit 830) of the light module 150 may also disposed on the flexible light guide film 810. However, the ultrasonic transducers on the flexible light guide film 810 can be omitted and disposed at other positions of the photoacoustic detector 100, which is not limited in the disclosure. In the present exemplary embodiment, the light illuminating the object 170 is relatively more uniform by using the flexible light guide film 810, so as to increase the quality of a photoacoustic image. On the other hand, an ultrasonic image with the same position corresponding to a photoacoustic image can be obtained via the ultrasonic transducer on the flexible light guide film 810.

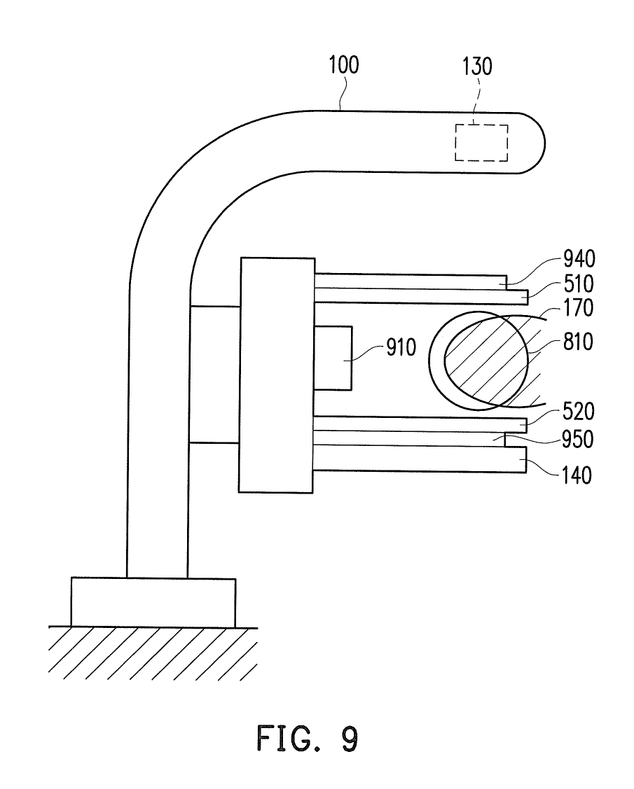

FIG. 9 is a schematic diagram illustrating the using of a flexible light guide film and a board-shape ultrasonic transducer according to an exemplary embodiment.

Referring to FIG. 9, in one exemplary embodiment, if the light module 150 includes the flexible light guide film, the sonic module 160 may includes a board-shape ultrasonic transducer. For example, the lighting unit 910 transmits light to illuminate the flexible light guide film 810. The flexible light guide film 810 guides the light so that the light illuminates the object 170. A board-shape ultrasonic transducer 940 is disposed at a side of the pressing board 510. In addition, a board-shape ultrasonic transducer 950 is disposed at a side of the pressing board 520. The board-shape ultrasonic transducers 940 and 950 are used for transmitting sonic waves and receiving sonic wave signals to generate an ultrasonic image and a photoacoustic image.

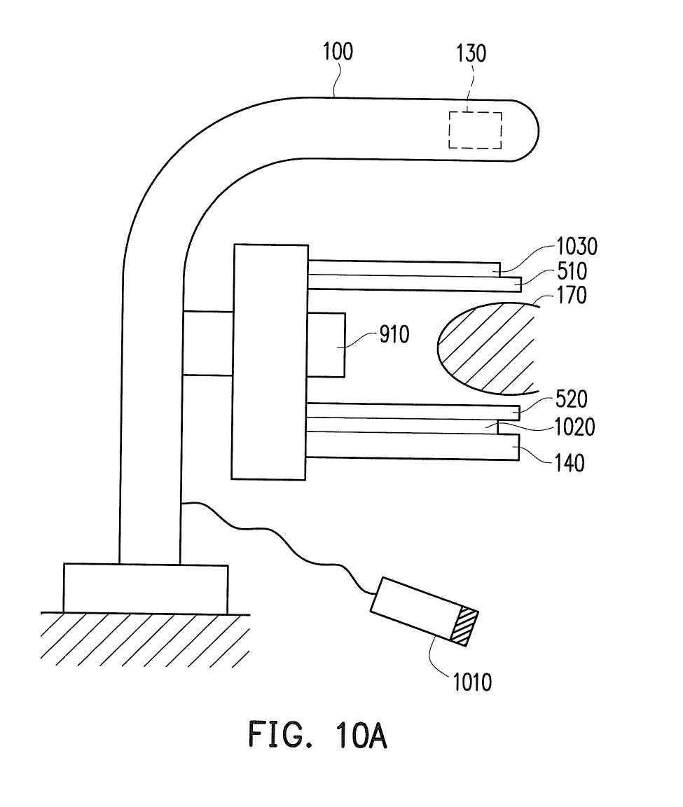

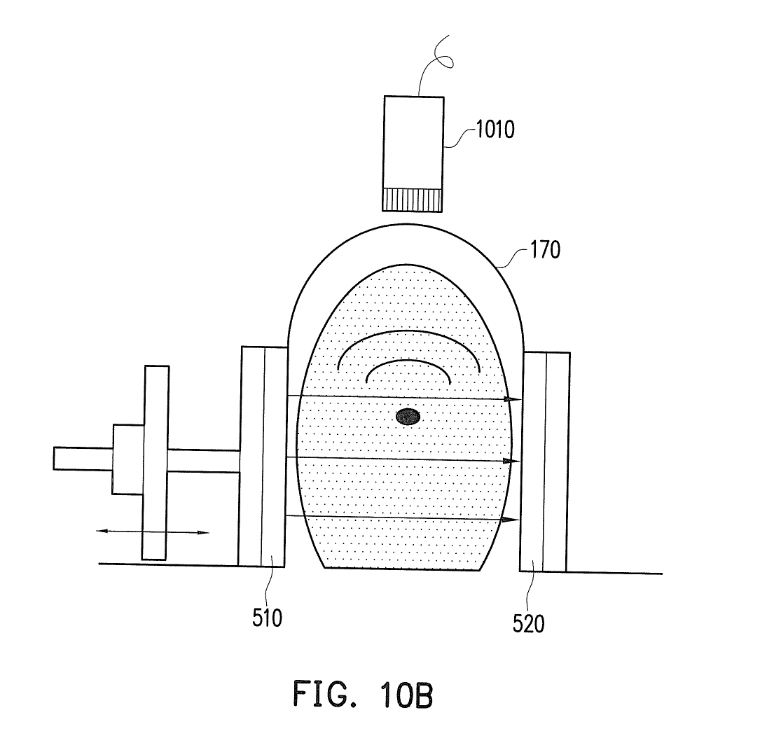

FIGS. 10A and 10B are schematic diagrams illustrating the using of an ultrasonic probe according to an exemplary embodiment.

Referring to FIGS. 10A and 10B, in one exemplary embodiment, the sonic module 160 is implemented as an ultrasonic probe 1010. The difference compared to the exemplary embodiment illustrated in FIG. 9 is that an optical board 1030 is disposed at a side of the pressing board 510, and an optical board 1020 is disposed at a side of the pressing board 520. The optical boards 1020 and 1030 include one or a plurality of lighting unit or light guide. The light illuminating the object 170 is more uniform due to the optical boards 1020 and 1030. In the exemplary embodiment illustrated in FIG. 10A, the lighting unit 910 is also capable of providing light. However, the lighting unit 910 can be omitted, and only the optical boards 1020 and 1030 provide the light in another exemplary embodiment, which is not limited in the disclosure. On the other hands, the ultrasonic probe 1010 receives the sonic wave signal generated due to the thermoelastic effect to generate a photoacoustic image. In addition, the ultrasonic probe is capable of transmitting a sonic wave and receiving the sonic wave signal generated due to the sonic wave to generate an ultrasonic image. In the exemplary embodiments illustrated in FIGS. 10A and 10B, medical personnel may generate a 3-dimensional (3D) ultrasonic image and a 3D photoacoustic image by moving the ultrasonic probe 1010. And, the medical personnel may also do biopsy at the same time aside.

In another exemplary embodiment, the optical boards 1020 and 1030 may be replaced by a photoacoustic board or a board-shape ultrasonic transducer, which is not limited in the disclosure.

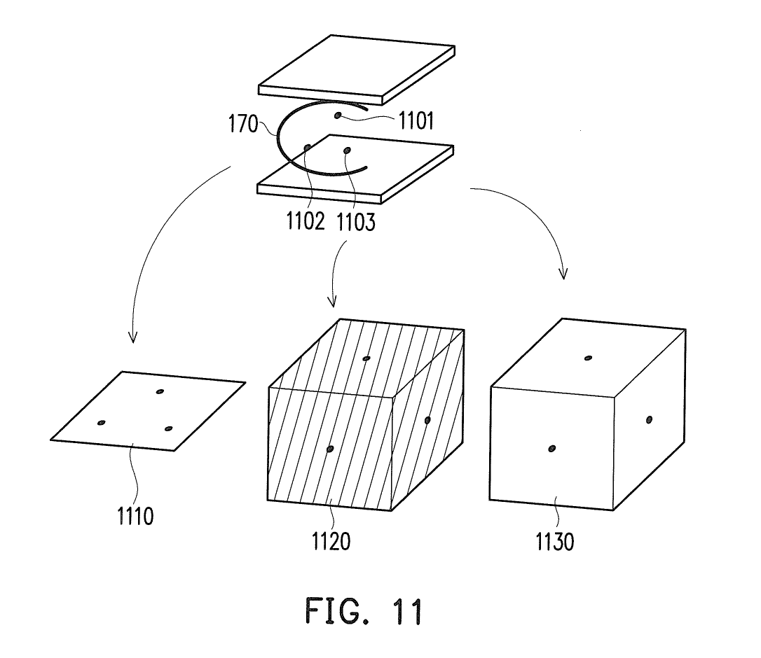

FIG. 11 is a schematic diagram illustrating a X-ray image, an ultrasonic image and a photoacoustic image according to an exemplary embodiment.

Referring to FIG. 11, assume the object 170 has calcified points (also referred as calcified tissue) 1011˜1103. The calcified points 1101˜1103 are projected on a 2-dimensional X-ray image 1110 via the X-ray transmitter 130 and the X-ray receiver 140. On the other hand, the calcified points 1101˜1103 are projected on 3D ultrasonic image 1120 (which has some speckle noises) via the sonic module 160. The calcified points 1101˜1103 are also projected on 3D photoacoustic image 1130 via the sonic module 160 and the light module 150. Medical personnel may obtain image information with same positions but different types (i.e. X-ray, ultrasonic, and photoacoustic) by comparing the X-ray image 1110, the ultrasonic image 1120 and the photoacoustic image 1130. Therefore, the existence of the calcified points 1101˜1103 is detected out more accurately.

The previously described exemplary embodiments of the present disclosure have the advantages aforementioned, wherein the advantages aforementioned not required in all versions of the disclosure.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

What is claimed is:

1. A photoacoustic detector for detecting an object, the photoacoustic detector comprising:
a X-ray transmitter, configured to transmit a X-ray to irradiate the object;
a X-ray receiver, configured to receive an image beam generated after the object is irradiated by the X-ray;
a light module, configured to provide light for illuminating the object to generate a first sonic wave signal; and
a sonic module, configured to receive the first sonic wave signal, transmit a sonic wave toward the object, and receive a second sonic wave signal generated after the object is interacted with the sonic wave.

2. The photoacoustic detector according to the claim 1, wherein the light module comprises a plurality of optical units, the optical units provide the light, the sonic module comprises a plurality of ultrasonic transducers, and the optical units and the ultrasonic transducer are disposed on a photoacoustic board in an array arrangement, wherein the optical units comprise a plurality of lighting units, a plurality of light guides or a combination thereof.

3. The photoacoustic detector according to the claim 2, wherein the optical units and the ultrasonic transducers are disposed on the photoacoustic in a chessboard arrangement, a random arrangement or a line arrangement.

4. The photoacoustic detector according to the claim 2, wherein the optical units comprise at least one lens and at least one actuator, the light passes through the at least one lens to illuminate the object, and the at least one actuator is configured to change a position of the lens to change an angle of the light transmitted from at least one of the lighting unit.

5. The photoacoustic detector according to the claim 2, wherein one of the ultrasonic transducers is a transparent ultrasonic sensor for being passed by the light transmitted from the lighting unit.

6. The photoacoustic detector according to the claim 2, further comprising a fixing unit, configured to fix the object,
wherein the fixing unit comprises a first pressing board and a second pressing board, and the photoacoustic board is disposed at a side of the first pressing board.

7. The photoacoustic detector according to the claim 1, wherein the light module comprises a lighting unit and a flexible light guide film, the lighting unit is configured to transmit the light, and the flexible light guide film is configured to cover the object and to guide the light.

8. The photoacoustic detector according to the claim 7, wherein the sonic module comprises a plurality of ultrasonic transducers, and the ultrasonic transducers are disposed on the flexible light guide film.

9. The photoacoustic detector according to the claim 7, wherein the sonic module comprises a board-shape ultrasonic transducer, configured to receive the first sonic wave signal, transmit the sonic wave and receive the second sonic wave signal,
wherein the photoacoustic detector further comprises a fixing unit, configured to fix the object, the fixing unit comprises a first pressing board and a second pressing board, and the board-shape ultrasonic transducer is disposed at a side of the first pressing board.

10. The photoacoustic detector according to the claim 1, wherein the sonic module comprises an ultrasonic probe, configured to receive the first sonic wave signal, transmit the sonic wave, and receive the second sonic wave signal,
wherein the photoacoustic detector further comprises a fixing unit, configured to fix the object, the fixing unit comprises a first pressing board and a second pressing board, the light module comprises a optical board, and the optical board is disposed at a side of the first pressing board.

11. The photoacoustic detector according to the claim 1, wherein the light module comprises an optical unit, the optical unit provides the light, and the sonic module comprises a ultrasonic transducer, configured to receive the first sonic wave signal, transmit the sonic wave, and receive the second sonic wave signal,
wherein the photoacoustic detector further comprises a fixing unit, configured to fix the object, the fixing unit comprises a first pressing board and a second pressing board, and the optical unit and the ultrasonic transducer are disposed on the first pressing board.

12. The photoacoustic detector according to the claim 1, wherein the sonic module comprises an ultrasonic transducer, configured to receive the first sonic wave signal, transmit the sonic wave and receive the second sonic wave signal,
wherein the ultrasonic transducer is a capacitive micromachined ultrasonic transducer, a polyvinylidene fluoride transducer, a CdZnTe transducer, a piezoelectric transducer, a piezoelectric micro-machined ultrasonic transducer, or a combination thereof.

13. The photoacoustic detector according to the claim 1, wherein the light module comprises a lighting unit, and the light uniting is a solid-state light source or a diode laser.

14. A photoacoustic board, comprising:
a plurality of optical units; and
a plurality of ultrasonic transducers,
wherein the optical units and the ultrasonic transducers are disposed on the photoacoustic board in an array arrangement.

15. The photoacoustic board according to the claim 14, wherein the optical units comprise a lighting unit, a light guide or a combination thereof.

16. The photoacoustic board according to the claim 15, wherein the optical units further comprise a lens and an actuator, and the actuator is configured to change a position of the lens to change an angle of the light transmitted from the light unit.

17. The photoacoustic board according to the claim 15, wherein one of the ultrasonic transducers is a transparent ultrasonic sensor for being passed by the light transmitted from the lighting unit.

18. A detector, comprising:
a photoacoustic board, comprising at least one optical unit and at least one ultrasonic transducer,
wherein the at least one optical unit is configured to provide light to illuminate an object to generate a first sonic wave signal,
wherein the at least one ultrasonic transducer is configured to receive the first sonic wave signal for generating a photoacoustic image, to transmit a sonic wave toward the object and to receive a second sonic wave signal generated after the object is interacted with the sonic wave for generating an ultrasonic image.
